(19) United States
(12) Patent Application Publication
(10) Pub. No.: US 2025/0195532 A1
(43) Pub. Date: Jun. 19, 2025

(54) NOVEL PHOSPHOINOSITIDE 3-KINASE (PI3K) INHIBITOR, COMPOSITIONS COMPRISING THE SAME, METHODS OF MAKING, AND METHODS OF TREATING A DISEASE

(71) Applicant: The Curators of the University of Missouri, Columbia, MO (US)

(72) Inventors: Kun CHENG, Overland Park, KS (US); Maryam NAKHJIRI, Olathe, KS (US); Umar-Farouk Wumbei MAMANI, Kansas City, MO (US); Pratikkumar Vinodchandra PATEL, Scarborough, ME (US)

(21) Appl. No.: 18/845,807

(22) PCT Filed: Mar. 10, 2023
(86) PCT No.: PCT/US2023/064190

§ 371 Date: Sep. 10, 2024

Related U.S. Application Data
(60) Provisional application No. 63/318,579, filed on Mar. 10, 2022.

(51) Int. Cl. A61K 31/5377; C07D 471/04

(52) U.S. Cl. CPC A61K 31/5377; C07D 471/04

(57) ABSTRACT

Aspects disclosed herein relate to compositions comprising novel phosphoinositide 3-kinase (PI3K) inhibitors, and methods of making the novel PI3K inhibitors. Further, various aspects relate to one or more methods of treating a disease using the compositions disclosed herein, including targeted delivery of the novel PI3K inhibitors disclosed herein.

### BACKGROUND OF THE INVENTION

Phosphoinositide 3-kinases (PI3Ks) are lipid enzymes that phosphorylate the 3′-hydroxyl group (OH) of the inositol ring of phosphoinositides (PIP) to convert PIP₂ to PIP₃, which is responsible for cell cycle progression, cell growth, cell survival and cell apoptosis (Tai, W. et al., Mol. Pharmaceutics 2011, 8, 901-912). Thus, it has been established that PI3Ks play a pivotal role in cell growth, proliferation, and survival (Engelman, J. A. et al., Nat. Rev Genet. 2006, 7(8), 606-19), and PI3Ks have been divided into three classes (I, II, and III) according to their different structural features and in vitro lipid substrate specificities (Wymann, M. P. et al., Biochim. Biophys. Acta 1998, 1436 (1-2), 127-50; and Marone, R. et al., Biochim. Biophys. Acta 2008, 1784 (1), 159-85). Class 1 PI3Ks can be further divided into two subclasses (class IA p110α/β/δ and class IB p110γ). Particularly, PI3K isoforms (p110α/β/δ) have been found to possess oncogenic potential through gain-of-function mutations or gene amplifications (Vogt, P. K. et al., Virology 2006, 344 (1), 1341-8). A negative regulator of the PI3K signaling pathway, phosphatase and tensin homologue (PTEN), has long been reported to be inactive in most human cancers, including prostate cancer (Ayala, G. et al., Clin. Cancer Res. 2004, 10 (19), 6572-8; Kreisberg, J. I. et al., Cancer Res. 2004, 64 (15), 5232-6; and Le Page, C. et al., Br. J. Cancer 2006, 94 (12), 1906-12).

For example, in PTEN-null prostate cancers, PI3Kβ(PI3K-p110β), rather than PI3KR, has been shown to contribute to tumorigenesis and androgen-independent progression (Jia, S. et al., Nature 2008, 454 (7205), 776-9). The chemical inhibition or genetic knockout of PI3Kβ is believed to prevent AR-dependent gene expression and tumor growth. 7-methyl-2-(4-morpholinyl)-9-[1-(phenylamino)ethyl]-4H-pyrido[1,2-a]pyrimidin-4-one (TGX-221) is a cell-permeable small molecule inhibitor of PI3Kβ. However, the therapeutic potential of TGX-221 in cancer therapy is halted due to its poor solubility (<1 mg/ml) and high lipophilicity (Log D_{7.4}=3.6), thus requiring an organic solvent for, for example intravenous administration of TGX-221. The organic solvent required causes significant toxicities that outweigh the therapeutic potential of TGX-221 (Jackson, S. P. et al., Nat. Med. 2005, 11 (5), 507-14). Therefore, there is a need for a more therapeutically effective analogue of TGX-221 that is soluble in aqueous solutions, is suitable for targeted delivery, and can be administered with minimal or at least reduced toxicity when compared to TGX-221.

### BRIEF DESCRIPTION OF THE DRAWING

This technology is described in detail herein with reference to the attached drawing figures, which are incorporated herein by reference, wherein:

FIG. 1 depicts the chemical structure of 9-(1-((2-aminoethyl)(phenyl)amino)ethyl)-7-methyl-2-morpholino-4H-pyrido[1,2-a]pyrimidin-4-one (CK-TGX-MN), in accordance with aspects described herein;

FIG. 2 depicts an example synthesis pathway for CK-TGX-MN, in accordance with aspects described herein;

FIG. 3A depicts a proton NMR (¹HNMR) of CK-TGX-MN;

FIG. 3B depicts pharmacokinetic analysis graphs of CK-TGX-MN in-vivo;

FIG. 4A depicts response curves of CK-TGX-MN and a control PI-103 against PI3K p110α/β/δ/γ;

FIG. 4B depicts response curves for CK-TGX-MN and a control PI-103 to calculate IC₅₀;

FIGS. 5A-5F depicts luminescence over concentration of TGX-221 and CK-TGX-MN, respectively, in different cancer cell lines to determine cellular uptake of CK-TGX-MN compared to TGX-221;

FIG. 6A depicts a PI3K pathway in a cell;

FIG. 6B depicts an example CK-TGX-MN conjugate, in accordance with aspects described herein;

FIG. 7A depicts an example general dipeptide conjugate of CK-TGX-MN, in accordance with aspects described herein;

FIG. 7B depicts an example dipeptide conjugates of CK-TGX-MN based on the general dipeptide conjugate shown in FIG. 7A, in accordance with aspects described herein;

FIG. 8 depicts an example targeting polypeptide conjugate of CK-TGX-MN for targeting prostate cancer, in accordance with aspects described herein; and

FIG. 9 depicts an example targeting pathway of the example targeting polypeptide shown in FIG. 8.

### DETAILED DESCRIPTION OF THE INVENTION

Certain aspects of the disclosure herein relate to compositions comprising one or more phosphoinositide 3-kinase (PI3K) inhibitors, and methods of making the PI3K inhibitors. Various aspects relate to a method of treating a disease using the compositions disclosed herein.

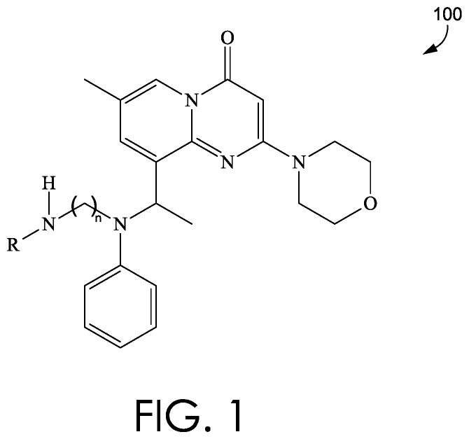

Generally, the disclosure herein relates to a novel PI3K-p110β inhibitor CK-TGX-MN, whose chemical structure 100 is shown in FIG. 1, where “n” is an integer between 1 and 6. The R-group in the chemical structure 100 represents any one of a hydrogen atom (H), an amino acid, a polypeptide, a dipeptide, a peptide chain, a targeting compound, a targeting moiety, a small molecule, and the like. CK-TGX-MN is a TGX-221 analogue that has surprisingly high stability and solubility in aqueous solutions, which may make it suitable for oral administration with relatively good bioavailability. CK-TGX-MN has been proven to have comparable effectiveness in inhibition of the PI3K-p110β pathway as TGX-221.

Throughout the description provided herein short hand is used for units of measurement. This short hand is used consistent with the International System of Units (SI). For example nM means nanomolar. Unless explicitly stated otherwise the use of ‘M’ in a unit of measure means moles or molar.

### Synthesis of CK-TGX-MN

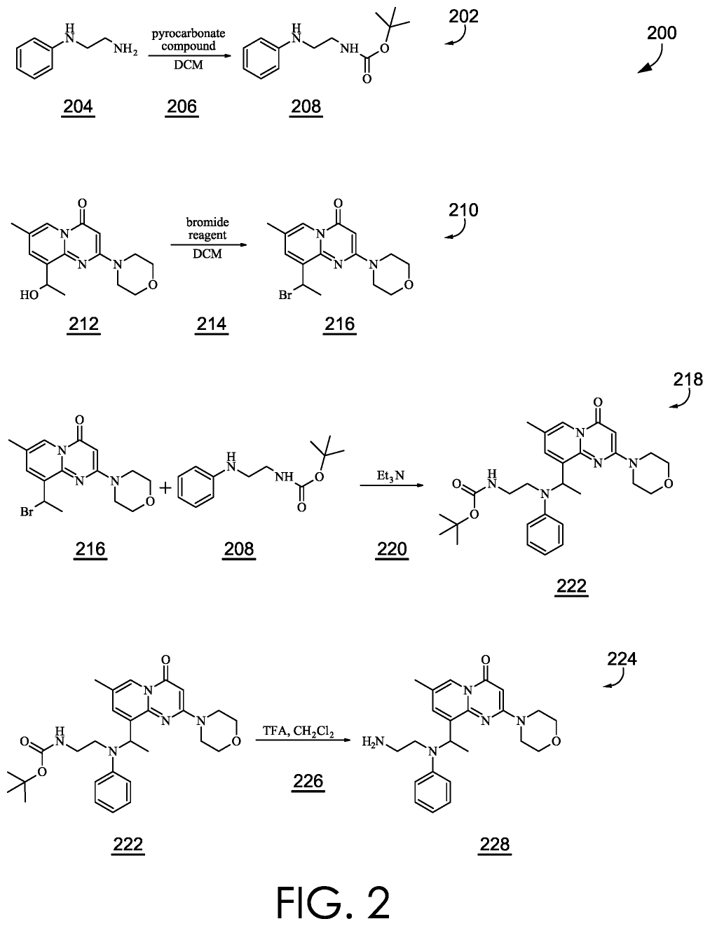

In other aspects, a method 200 for synthesizing the novel PI3K-p110β inhibitor CK-TGX-MN is disclosed herein, as shown in FIG. 2. As shown in step 202, an amount of N¹-phenylethane-1,2-diamine (C₈H₁₂N₂) compound 204 may be dissolved in a suitable solvent, such as, for example, dichloromethane (DCM, CH₂Cl₂), and stirred while cooling the solution at 0° C. Then, an amount of a suitable pyrocarbonate compound 206 (e.g., di-tert-butyl decarbonate (C₁₀H₁₈O₅)) also dissolved in the suitable solvent may be gradually added to the cooled solution of compound 204. The solution of compound 204 and the pyrocarbonate compound 206 may be cooled and stirred for a set amount of time (e.g., 30 minutes, 60 minutes, 70, minutes, and the like), and may be brought to room temperature while continuing to stir the reaction mixture for another set amount of time (e.g., between 2 hours and 24 hours, between 4 hours and 20 hours, between 4 hours and 16 hours, between 4 hours and 12 hours, or overnight). The reaction was confirmed by thin layer chromatography (TLC). The organic layer formed, was washed with 5% citric acid solution for a number of times (e.g., once, twice, three times, four times, five times, etc.) followed by a brine wash. The reaction mixture was dried over sodium sulfate (Na₂SO₄) and the extract was concentrated in vacuo to yield an amount of tert-butyl (2-(phenylamino)ethyl)carbamate (C₁H₁₈N₂O₂) compound 208, which may appear as a pearl white powder.

Further, as shown in step 210, an amount of 9-(1-hydroxyethyl)-7-methyl-2-morpholino-4H-pyrido[1,2 a]pyrimidin-4-one (C₁₅H₁₉N₃O₃) compound 212 may be dissolved in a suitable solvent (e.g., dichloromethane (CH₂Cl₂)). The solution may be subsequently cooled to 0° C. and a bromide reagent 214 (e.g., phosphorus tribromide(PBr₃)) suitable for conversion of the secondary alcohol in compound 212 to an alkyl bromide may be gradually added and stirred for a set amount of time (e.g., between 2 hours and 72 hours, between 4 hours and 48 hours, between 4 hours and 24 hours, between 4 hours and 16 hours, or overnight). The reaction mixture was then diluted in dichloromethane and was neutralized with pyridine followed by one or more washes with dichloromethane (THF, (CH₂)₄O). The filtrate was condensed in vacuo and the crude solid was washed with tetrahydrofuran followed by centrifuging in 9000 rpm for at least a set amount of time (e.g., 2 minutes, 5, minutes, 10 minutes, and the like) to yield 9-(1-bromoethyl)-7-methyl-2-morpholine-4H-pyrido[1,2 a]pyrimidin-4-one (C₁₅H₁₉N₃O₂Br₁) compound 216, which may appear as a yellow powder. The reaction was confirmed by TLC.

At step 218, an amount of dried compound 208 may be dissolved in a suitable solvent (e.g., dichloromethane (CH₂Cl₂)). The solution of compound 208 may then be cooled to 0° C. and an amount of triethyl amine (Et₃N, C₆H₁₅N) compound 220 may be gradually added while stirring the solution of compound 208. Additionally, an amount of dried compound 216 may be dissolved in a suitable solvent (e.g., dichloromethane) and gradually added to the cooled solution of compound 208 and triethyl amine compound 220. The reaction mixture may be refluxed at a temperature above room temperature (e.g., 50° C.) for at least 24 hours under nitrogen to yield tert-butyl(2-((1-(7-methyl-2-morpholino-4-oxo-4Hpyrido[1,2-a]pyrimidin-9-yl)ethyl)(phenyl)amino)ethyl)carbamate compound 222, which may appear as a light yellow powder. The end of the reaction may be regularly monitored by, for example, TLC and/or LCMS (liquid chromatography mass spectrometry).

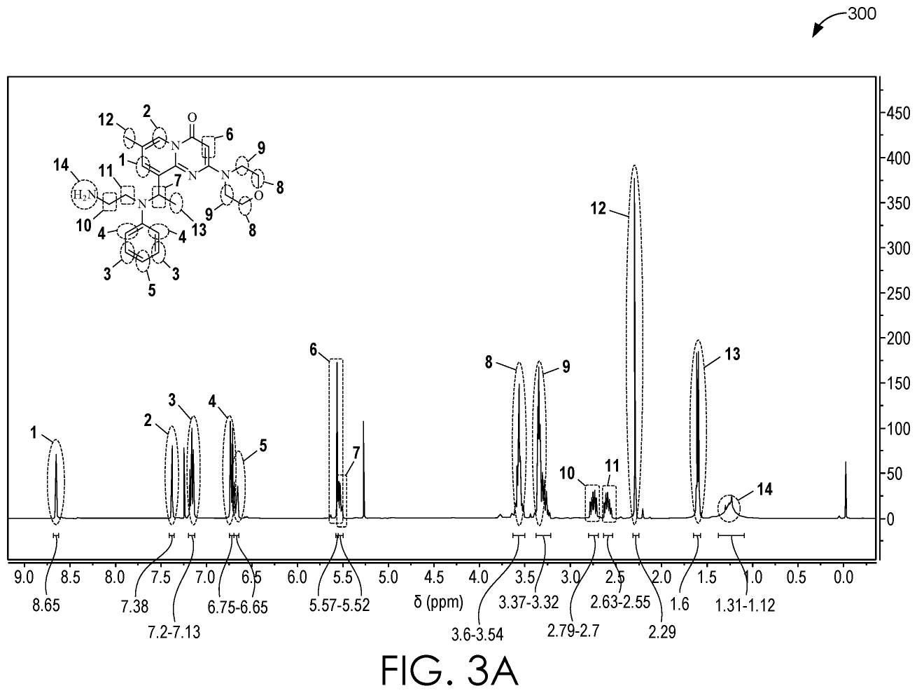

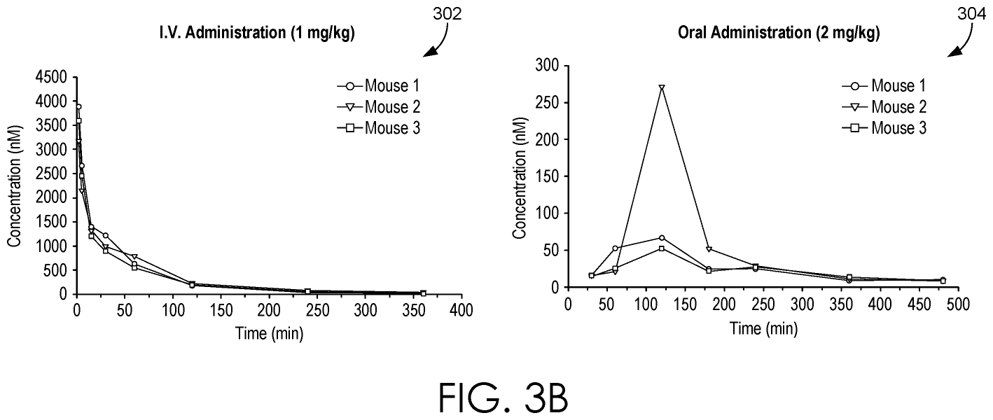

At step 224, CK-TGX-MN compound 228 may be formed by dissolving an amount of purified and dried compound 222 in a suitable solvent 226 (e.g. a solution of dichloromethane and trifluoroacetic acid (TFA, CF₃CO₂H)), and stirring the reaction mixture for a predetermined amount of time or until the reaction is deemed to be complete to yield CK-TGX-MN compound 228. The solvent was evaporated and co-evaporated with methanol (CH₃OH) followed by washing with dichloromethane and saturated sodium bi-carbonate (NaHCO₃). The reaction may be confirmed by TLC, and once the CK-TGX-MN compound 228 is purified, it may appear as a light yellow powder, the structure and purity of which may be confirmed by, for example, ¹HNMR yielding the ¹HNMR spectrum 300, as provided in FIG. 3A.

As shown in FIG. 3A, the peaks in, for example, an ¹HNMR spectrum 300 (¹HNMR 400 MHz, CDCl₃), represent groups of types of H atoms found in a compound, and the area under a peak is generally proportional to the number of H atoms in the group of the type of H atom that the peak represents. The chemical shift is the position of a peak on the 6 scale (in ppm). In other words, the position of a peak on an ¹HNMR spectrum indicates the type of H atom present in the structure and their proximity to one another. For instance, in the ¹HNMR spectrum 300, peak 1 found at ˜8.65 ppm represents a singlet (1H), peak 2 found at ˜7.38 ppm represents a singlet (1H), peak 3 found between ˜7.2-7.13 represents a multiplet (2H), peaks 4 and 5 found between ˜6.75-6.65 represent a multiplet (3H), peaks 6 and 7 found between ˜5.57-5.52 represent a multiplet (2H), peak 8 found between ˜3.6-3.54 represents a multiplet (4H), peak 9 found between ˜3.37-3.32 represents a multiplet (4H), peak 10 found between ˜2.79-2.70 represents a multiplet (2H), peak 11 found between ˜2.63-2.55 represents a multiplet (2H), peak 12 found at ˜2.29 represents a singlet (3H), peak 13 found at 1.6 represents a doublet (3H), and peak 14 found between ˜1.31-1.12 represents a broad singlet (2H).

### Aqueous Solubility of CK-TGX-MN

The United States Pharmacopeia (USP) provides the following definitions for aqueous solubility:

TABLE 1

| | Parts of Solvent | Solubility Range of |
|---|---|---|
| Descriptive term | Required | solute to |
| (solubility definition) | for 1 Part of Solute | solvent (mg/ml) |
| Very soluble (VS) | <1 | >1000 |
| Freely soluble (FS) | From 1 to 10 | 100-1000 |
| Soluble (S) | From 10 to 30 | 30-100 |
| Sparingly soluble (SPS) | From 30 to 100 | 10-30 |
| Slightly soluble (SS) | From 100 to 1000 | 1-10 |
| Very slightly soluble (VSS) | From 1000 to 10000 | 0.1-1 |
| Practically insoluble (PI) | >10000 | <0.1 |

First two columns include definitions of aqueous solubility from The United States Pharmacopeia, USP 30-NF 25, 2007 and USP 38 General Notices (third column is an equivalent conversion).

According to solubility tests in aqueous solutions, the aqueous solubility of CK-TGX-MN was found to be 4.63±0.17 mg/ml (11.36±0.41 mM), which is comparatively higher than the aqueous solubility of TGX-221, which was found to be 0.09±0.01 mg/ml (0.25±0.03 mM).

### Bioavailability of CK-TGX-MN

Bioavailability in Pharmacology is a measure of an amount of a drug substance that enters the circulation and becomes completely available for its intended purpose when introduced into the body. There are several delivery routes for drugs including intra-venous route (IV), oral route, subcutaneous route, rectal route, intramuscular route, vaginal route, inhaled route, and the like. Among these routes, the IV route is considered to have a 100% bioavailability and is used as a standard for deriving percent bioavailability through the other non-IV routes.

Bioavailability of CK-TGX-MN was determined in relation to a liquid formulation of CK-TGX-MN 1 mg/ml in 5% DMSO, 10%, and 85% aqueous solution. A dose amount of aqueous CK-TGX-MN was prepared for delivery through the IV route in-vivo. Additionally, a liquid formulation of CK-TGX-MN 2 mg/ml in 5% DMSO, 10%, and 85% aqueous solution was prepared for delivery of a dose amount of CK-TGX-MN through the oral route in-vivo. The pharmacokinetic (PK) studies were performed in mice. As shown in graph 302 of FIG. 3B, the depicted example mice had similar clearance of IV delivered CK-TGX-MN over time. Additionally, as shown in graph 304 of FIG. 3B, mouse 1 and mouse 3 had similar absorption and clearance of orally delivered CK-TGX-MN, while mouse 2 had a higher absorption and slower clearance of orally delivered CK-TGX-MN. Based on the PK studies described above, the oral bioavailability of CK-TGX-MN in-vivo was found to be ˜6.4%.

Efficacy of CK-TGX-MN compared to PI-103

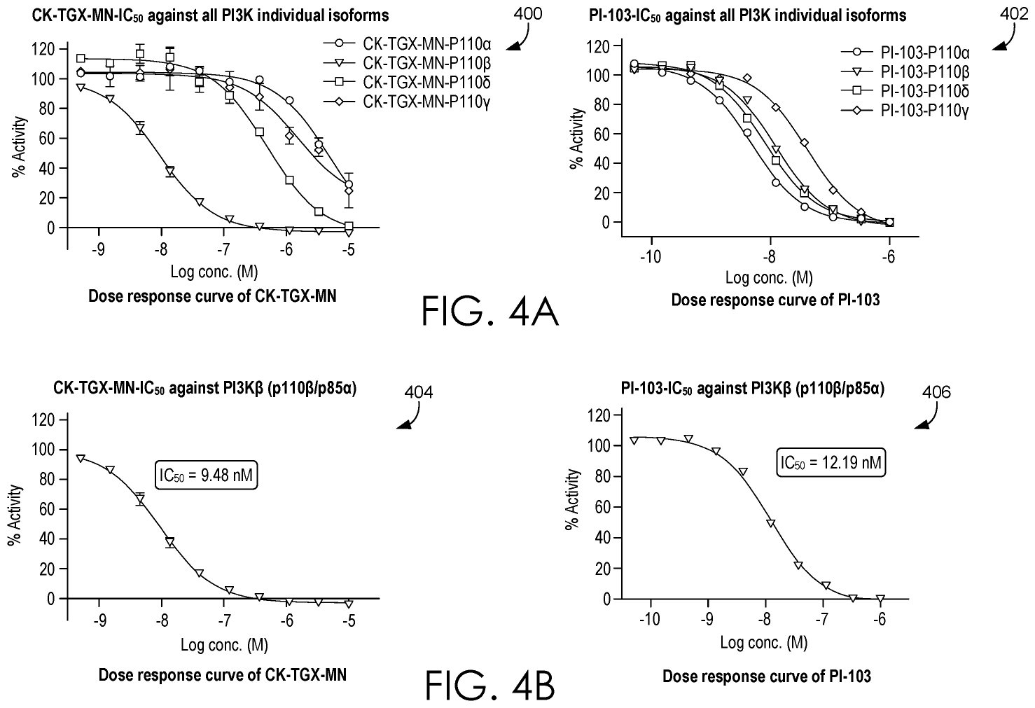

PI-103, a known inhibitor of PI3K pathways was used as a control to measure inhibitory properties of CK-TGX-MN. For example, FIG. 4A represents the inhibition of FIG. 4B represents the inhibition of PI3K p110α/β/γ isoforms by CK-TGX-MN compared to PI-103 as a function of % activity and concentration of CK-TGX-MN. As shown, in dose response curve graph 400, TGB-NH2 performs much better than PI-103 at inhibiting PI3K$ isoform, as shown in the dose response curve graph 402. Particularly, as shown in FIG. 4B in the dose response curve graph 404 (showing performance of CK-TGX-MN) and the dose response curve 406 (showing performance of PI-103) against PI3Kβ isoform, CK-TGX-MN has been found to have, for example an IC₅₀ of 9.48 nM, while PI-103 has an IC₅₀ of 12.19 nM. Thus, demonstrating that a lower concentration of CK-TGX-MN is required to deactivate PI3Kβ isoform pathway when compared to PI-103. In other words, a much lower concentration of CK-TGX-MN than PI-103 is more effective in inhibiting the PI3K0 isoform pathway.

### Cellular Uptake

Different cell lines were seeded, incubated, and washed prior to administering a dose solution of CK-TGX-MN. Cell lines tested included LNCaP cell line, which is a cell line with epithelial-like morphology that was isolated from a metastatic lymph node a lesion of human prostate cancer; C₄₋₂, which is a prostate cancer cell line derived from LNCaP cells that grows in the absence of androgens, yet responds to manipulation of androgen levels; GL261 cell line, which are murine glioma cells isolated from human brain; PC-3 cell line, which is a prostate cancer cell line that is typically used as a model of androgen-independent prostate cancer; TRAMP cell line, which are epithelial cells isolated from transgenic adenocarcinoma of the mouse prostate; and Hela cell line, which is an immortalized human cell line isolated from cervical cancer cells.

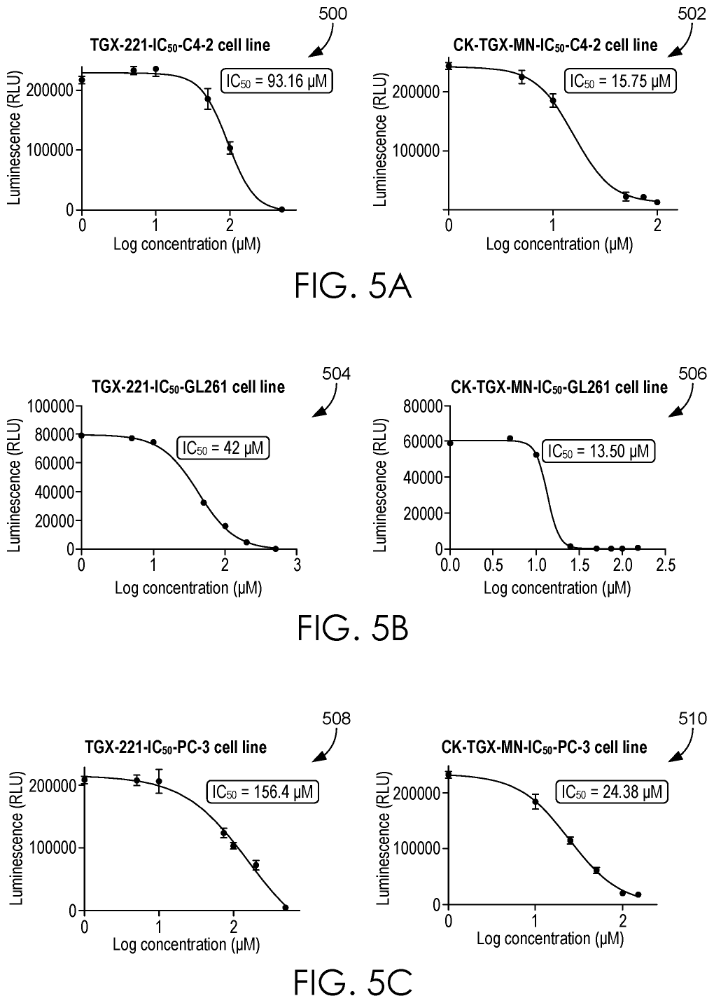

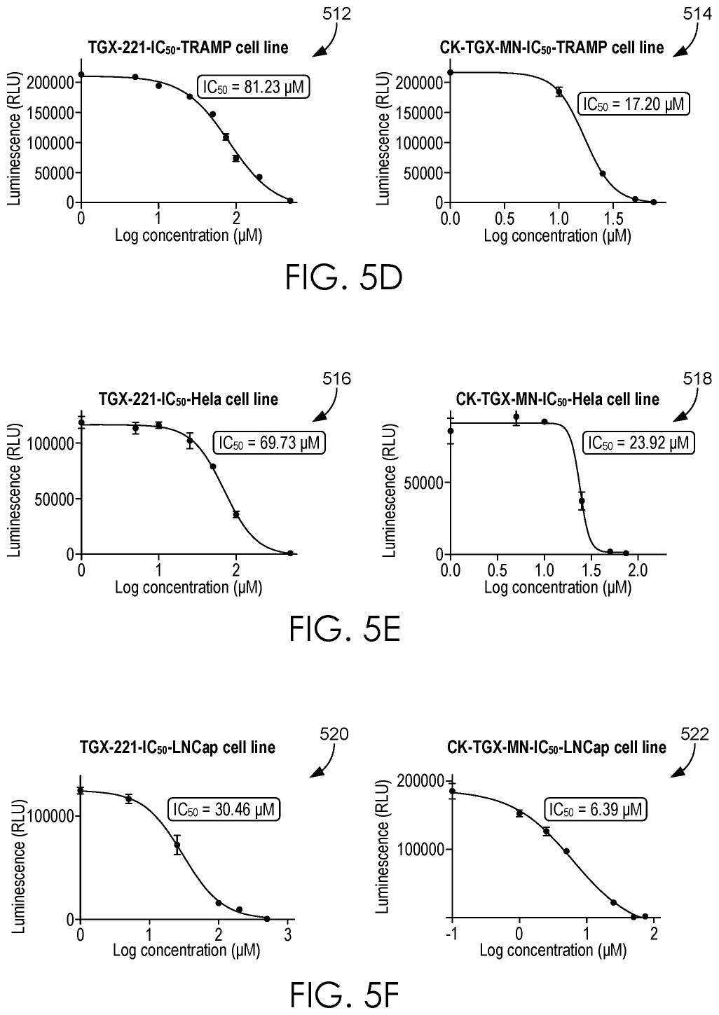

The cellular uptake was measured as a function of luminescence and concentration of CK-TGX-MN, by determining the IC₅₀ value. A decrease in luminescence observed translates to the efficacy of CK-TGX-MN in killing the cells of the respective cell culture. As shown in FIG. 5A, the cellular uptake of CK-TGX-MN and TGX-221 in C₄₋₂ cell line cells was measured. As shown in graph 502, it was found that CK-TGX-MN has an IC₅₀=15.75 μM, and as shown in graph 500, TGX-221 has an IC₅₀=93.16 μM. Thus, demonstrating that CK-TGX-MN has a greater cellular uptake than TGX-221 in C₄₋₃ cell line cells.

Similarly, as shown in FIG. 5B, the cellular uptake of CK-TGX-MN and TGX-221 in GL261 cell line cells was measured. As shown in graph 506, it was found that CK-TGX-MN has an IC₅₀=13.5 μM, and as shown in graph 504, TGX-221 has an IC₅₀=42 μM. Thus, demonstrating that CK-TGX-MN also has a greater cellular uptake than TGX-221 in GL261 cell line cells.

Moving on to FIG. 5C, the cellular uptake of CK-TGX-MN and TGX-221 in PC-3 cell line cells was measured. As shown in graph 510, it was found that CK-TGX-MN has an IC₅₀=24.38 μM, and as shown in graph 508, TGX-221 has an IC₅₀=156.4 μM. Thus, demonstrating that CK-TGX-MN has a greater cellular uptake than TGX-221 in PC-3 cell line cells.

Moving on to FIG. 5D, the cellular uptake of CK-TGX-MN and TGX-221 in TRAMP cell line cells was measured. As shown in graph 514, it was found that CK-TGX-MN has an IC₅₀=17.2 μM, and as shown in graph 512, TGX-221 has an IC₅₀=81.23 μM. Thus, demonstrating that CK-TGX-MN has a greater cellular uptake than TGX-221 in TRAMP cell line cells.

Moving on to FIG. 5E, the cellular uptake of CK-TGX-MN and TGX-221 in Hela cell line cells was measured. As shown in graph 518, it was found that CK-TGX-MN has an IC₅₀=23.92 μM, and as shown in graph 516, TGX-221 has an IC₅₀=69.73 μM. Thus, demonstrating that CK-TGX-MN has a greater cellular uptake than TGX-221 in Hela cell line cells.

Finally, moving on to FIG. 5F, the cellular uptake of CK-TGX-MN and TGX-221 in LNCaP cell line cells was measured. As shown in graph 522, it was found that CK-TGX-MN has an IC₅₀=6.39 μM, and as shown in graph 520, TGX-221 has an IC₅₀=30.46 μM. Thus, demonstrating that CK-TGX-MN also has a greater cellular uptake than TGX-221 in LNCap cell line cells.

Thus, based on the cellular uptake data, it can be seen that the therapeutic properties of CK-TGX-MN are significant for various cancer types.

### PI3K Pathway in Cancer

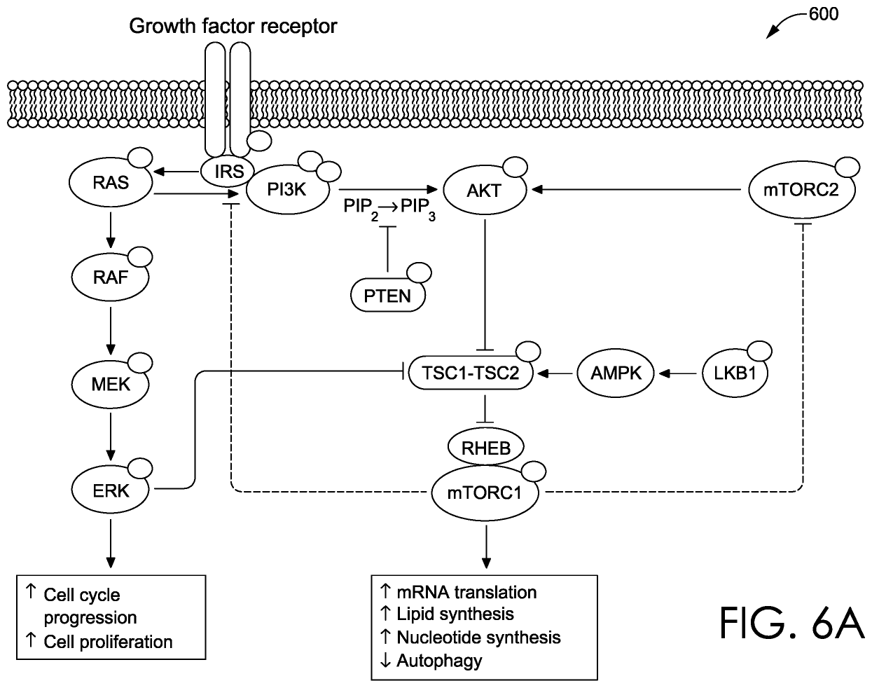

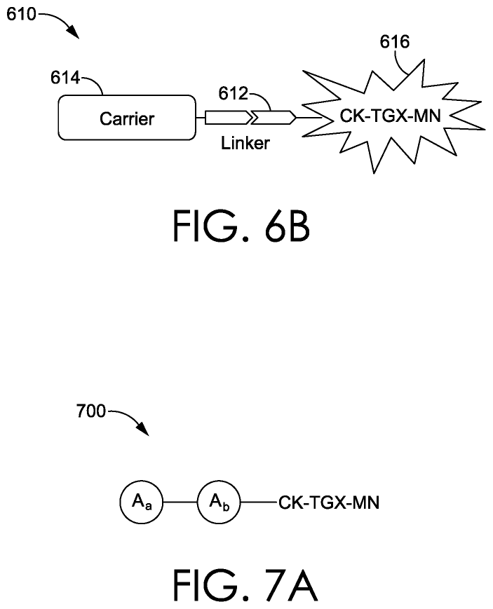

FIG. 6A depicts an example PI3K pathway 600 in accordance with aspects herein. PI3K/AKT/rapamycin (mTOR) signaling is one of the most important intracellular pathways, which has been found to at least regulate cell growth and angiogenesis. Activation of the PI3K/AKT/mTOR pathway causes tumor growth and resistance to anticancer therapies (Yang, J. et al., Molecular Cancer 2019, 18:26). Upon activation, PI3K catalyzes the phosphorylation of PtdIns(4, 5) P2 (PIP2) to produce PtdIns(3, 4, 5) P3 (PIP 3). A variety of signaling proteins, such as kinases AKT and PDK1 can bind to the lipid products of PI3K and thereby localize to the cell membrane to activate cell growth and cell survival pathways (Manning B. D., et al., Cell 2007, 129:1261-74). Phosphatase and tensin homologue deleted on chromosome 10 (PTEN) regulates the pathway by dephosphorylating PIP3 to PIP2 and thus prevents activation of downstream kinases (Hennesy B. T., et al., Nat. Rev. Drug Discov. 2005, 4: 988-1004). Therefore, in PTEN-deficient cancer, the main carcinogenic driving force is the overactivation of AKT caused by the loss of PTEN lipid phosphatase function (Papa A., et al., Cell 2014, 157: 595-610; Haddadi N., et al., Mol. Cancer 2018, 17: 37). Additionally, the RAS-RAF-MEK-ERK signaling pathway has been found to be interconnected with PIrK signaling (Castellano E., et al., Genes Cancer 2011, 2: 261-74).

### Targeted Delivery of CK-TGX-MN

Throughout the disclosure, the terms “peptide” and “polypeptide” are used interchangeably. Thus, unless specifically noted otherwise, “peptide” and “polypeptide” shall not limit one or the other, but shall be construed to have the same, broadest meaning.

Peptide drug conjugates involve the attachment, bonding, joining, or linking of various compounds. In some instances, the term a “linking moiety” is used to refer to these aspects. As such, “linking moiety” is meant to include any conventional linking moieties known to one skilled in the art that can covalently link two peptide sequences together, such as another amino acid residue, e.g., lysine. The linking moiety may also comprise aminohexaonic acid; (CH₂)₄; (CH₂)₅; (CH₂)₆; (CH₂)₇; (CH₂)₈; (CH₂)₉; polyethylene glycol chain (PEG) chain having the formula (OCH₂CH₂)ₓ, where “x” can be an integer between 4 and 182, between 4 and 150, between 4 and 100, between 4 and 50, between 4 and 35, between 4 and 30, between 4 and 24, between 4 and 23, between 4 and 20, between 4 and 10, between 4 and 9, between 5 and 9, and the like.

Furthermore, the term “linking moiety” may be used to describe the connection or joining of an agent to a polypeptide. The term “linking moiety” shall include any conventional attachment moiety known to one skilled in the art that can covalently link a therapeutic agent to the polypeptide, including amino acid residues, small molecules, peptides, proteins, nucleic acids, polymers, lipids, inorganic nanoparticles, imaging agents, and radioisotopes. A “targeting moiety” as used herein, may include a small molecule, an amino acid, or a polypeptide having affinity to a cell antigen or receptor expressed in a specific type of cancer cell, or a specific type of cell, depending on the treatment type.

In certain aspects, an imaging agent may be attached or included with a targeting composition or system. An imaging agent shall include any suitable imaging agent known in the art.

FIG. 6B illustrates a CK-TGX-MN conjugate 610 that may be used for targeted delivery of CK-TGX-MN compound 616, which as provided above, is a novel PI3K-p110β inhibitor. In example aspects, the CK-TGX-MN compound 616 can be conjugated with an amino acid, a dipeptide, a peptide or peptide chain (not shown), a linker 612 (e.g., linking moiety), and/or a carrier 614, or a combination thereof. In other aspects, the linker 612 may be comprised of a dipeptide or a peptide chain that forms a targeting substrate (e.g., a targeting peptide, a targeting polypeptide (i.e., comprised of more than one amino acid), a targeting amino acid, a targeting dipeptide, and the like). The linker 612 may include a cleavable linker, a non-cleavable linker. If cleavable, the linker may be cleavable by a protease, a hydrolysis reaction, or redox (oxidation-reduction) reactions, and the like. In some aspects, the carrier 614 may include an aptamer, a peptide sequence, small molecules, a protein sequence, an antibody, and the like.

Some aspects described herein may be used as a therapeutic drug or peptide-drug conjugate. The therapeutic uses of the drug or peptide-drug conjugates described herein, may be used in targeted cancer therapies to treat, for example, cancerous tumors, and more specifically, PTEN driven tumor cells such as, for example, in prostate cancer, lung cancer, colon cancer, breast cancer, gastric cancer, melanoma, glioblastoma, and the like. It is contemplated that as mentioned briefly above, the targeting therapies can be used for numerous types of cancers that include the deregulation of the PI3K pathway in cancerous cells. However, for illustrative purposes, but not limited to, therapeutic use of embodiments described herein are provided in the context of targeting pathways for prostate cancer.

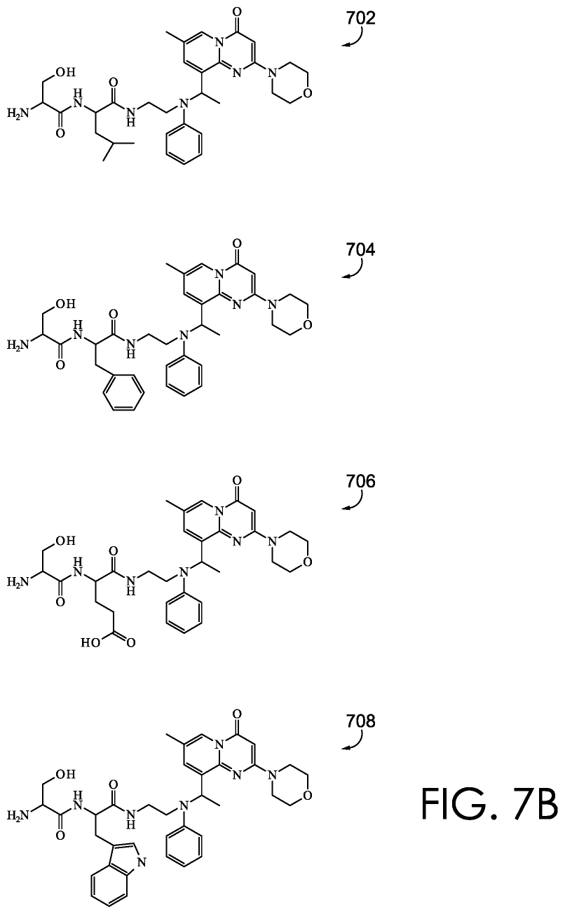

For example, according to aspects of the present invention, the targeting conjugates of CK-TGX-MN for targeting prostate cancer cells may comprise a peptide or a polypeptide conjugated to CK-TGX-MN compound, the peptide or polypeptide including a specific sequence of amino acids. For example, the targeting conjugate may include a dipeptide that is capable of being internalized by the prostate cancer cell (e.g., membrane peptide transporter), and is cleavable by a protein kinase (e.g., dendritic cell-derived protein kinase (DPK)) once inside the cell to free up CK-TGX-MN inside the cell. The dipeptide may be linked, directly or indirectly, to the CK-TGX-MN molecule in accordance with aspects herein. For example, as shown in FIG. 7A, the targeting CK-TGX-MN conjugate 700 may be a dipeptide composed of Aₐ and A_{b} amino acids conjugated to CK-TGX-MN. FIG. 7B, shows some example dipeptides that may be conjugated to CK-TGX-MN. For example, the dipeptide may include, for example, a serine-leucine (SL) dipeptide 702, a serine-phenylalanine (SF) dipeptide 704, a serine-glutamic acid (SE) dipeptide 706, a serine-tryptophan (SW) dipeptide 708, and the like.

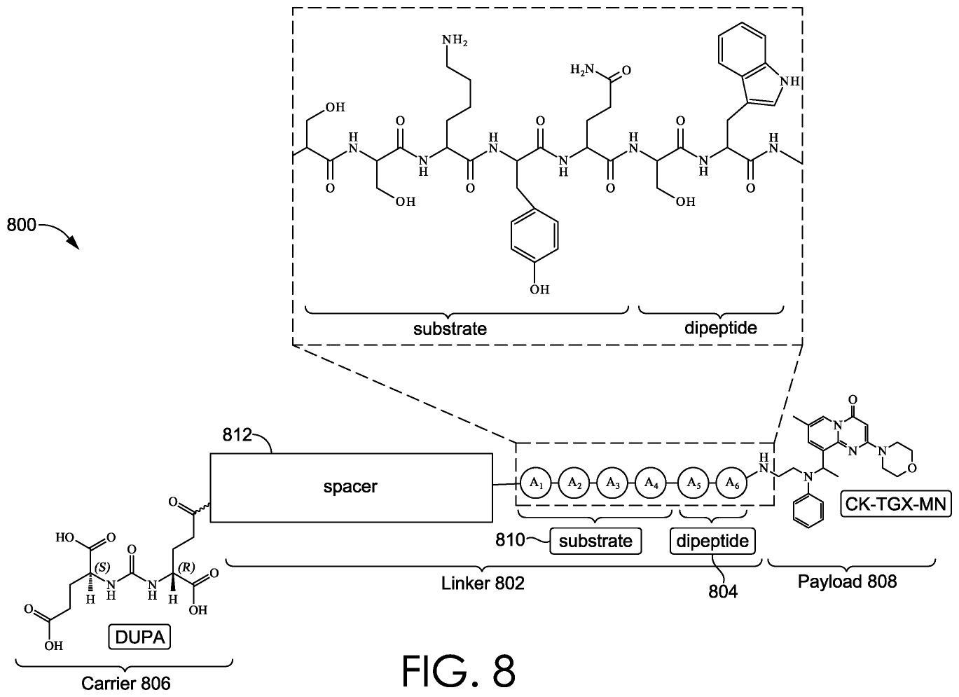

FIG. 8 depicts another example targeting conjugate 800 of CK-TGX-MN. Generally, the CK-TGX-MN conjugate 800 includes payload 808 and linker 802. The payload 808 includes CK-TGX-MN. Aspects of linker 802 can include a dipeptide 804, a targeting substrate 810, a spacer 812, or any combination thereof. Dipeptide 804 may be any suitable amino acid pair in some aspects. For example, in specific aspects dipeptide 804 includes a serine-leucine (SL) dipeptide (e.g., 702 of FIG. 7B), a serine-phenylalanine (SF) dipeptide (e.g., 704 of FIG. 7B), a serine-glutamic acid (SE) dipeptide (e.g., 706 of FIG. 7B), or a serine-tryptophan (SW) dipeptide (e.g., 708 of FIG. 7B).

Additionally, or alternatively, the linker 802 may comprise a polypeptide sequence forming, for example, a substrate 810 for an extracellular or intracellular protein. For example, in some embodiments substrate 810 is a substrate for Prostate Specific Antigen (PSA). PSA is an androgen-regulated protease that belongs to the serine protease family. PSA is frequently abundant in semen and is commonly used as a diagnostic indicator of prostate cancer. The substrate 810 of PSA may be comprised of an amino acid sequence that is cleavable by PSA when the substrate 810 is in the vicinity of PSA. For example, as depicted, the substrate 810 may be comprise amino acids serine (S), lysine (K), tyrosine (Y), and glutamine (Q) forming the sequence SSKYQ. As depicted, substrate 810 may be covalently bonded to dipeptide 804. As discussed above and in relation to FIG. 7B, the dipeptide 804 may be, for example, any one of, for example, SL, SF, SE, SY, SQ, SK, SD, SS, or SW (SW is depicted in FIG. 8).

Additionally, or alternatively, the linker 802 can include a molecular spacer 812. The molecular spacer 812 may be a polymer of polyethylene glycol (PEG), other similar polymers, a substituent alkyl group, an alkyl chain, or any other suitable molecular spacer. The molecular spacer 812 (e.g., PEG) may be linked (e.g., covalently bounded) to the substrate 810 or the dipeptide 804. The molecular spacer 812 may be incorporated through, for example, known “click chemistry” reactions. For example, the molecular spacer 812 can be incorporated using an azide-alkyne reaction to form a triazole linkage. In a particular aspect, molecular spacer 812 is a polymer of PEG. The PEG polymer chain may aid in elevation of drug accumulation in the targeted tumor, reduce non-specific protein binding, and prolong blood circulation time. The PEG chain can have the formula (OCH₂CH₂)ₓ, where “x” can be an integer between 4 and 182, between 4 and 150, between 4 and 100, between 4 and 50, between 4 and 35, between 4 and 30, between 4 and 24, between 4 and 23, between 4 and 20, between 4 and 10, between 4 and 9, between 5 and 9, and the like.

As depicted in FIG. 8, the CK-TGX-MN conjugate 800 may further comprise a carrier substrate 806. The carrier substrate 806 may include compounds that, at least partially, facilitate targeting of the CK-TGX-MN conjugate 800. For example, the carrier substrate 806 can include compounds that are substrates for cellular membrane proteins. The particular carrier substrate may be intentionally selected to bind with a cellular membrane protein that is overly expressed or otherwise specific to the targeted cancerous cells. As depicted, and in the continuing context of prostate cancer, the carrier substrate 806 can be a substrate for Prostate Specific Membrane Antigen (PSMA). Particularly, PSMA has been identified to be an effective targeting moiety in prostate cancer therapies because PSMA is overexpressed on prostate epithelial cells in nearly all prostate cancers and all tumor stages (100 to 1000 fold higher) (Barve et al., J. Control Release 2014; M. R. A. Pillai et al., Nuclear Medicine and Biology 2016). The carrier substrate 806 may for example, be 2-[3-(1,3-dicarboxypropyl)ureido]pentanedioic acid (DUPA), first synthesized by Kozikowski group in 2001 (Gourni et al., Molecules 2017; Jin et al., Urology 2016). DUPA has been found to have a high affinity for PSMA. Thus, DUPA may be a preferred carrier for CK-TGX-MN targeting composition intended to treat prostate cancers.

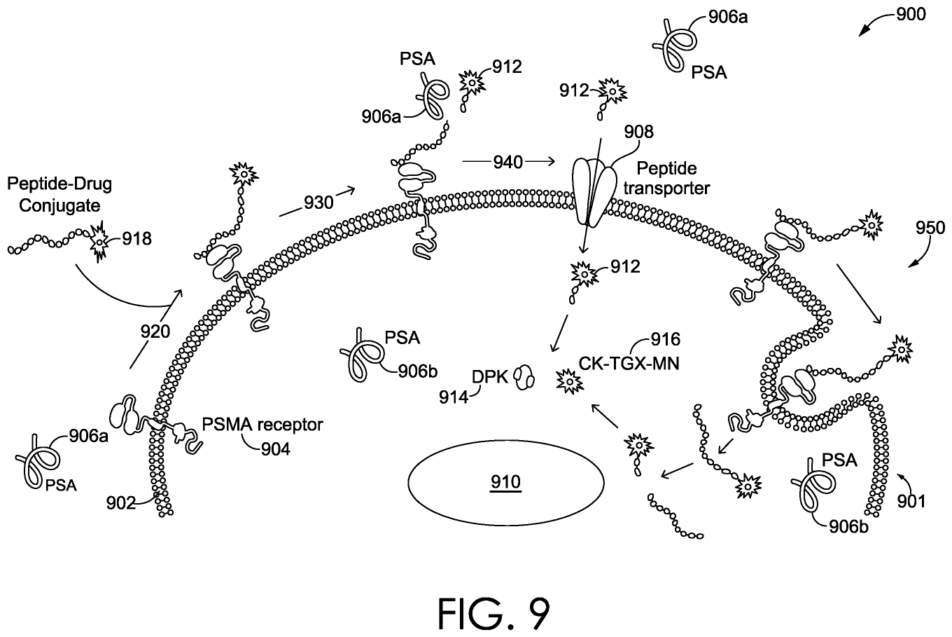

FIG. 9 depicts example 900 of selective portions of two targeting pathways in cell 901 having a nucleus 910. The example targeting polypeptide CK-TGX-MN 918 may be the CK-TGX-MN conjugate 800 described in relation to FIG. 8. As shown, the PSMA receptor 904 sits in a cell membrane 902. As depicted, PSA 906 may be abundant both extracellularly (PSA 906a) and intracellularly (PSA 906b). At step 920, the carrier (e.g., carrier 806 of FIG. 8) of the targeting polypeptide CK-TGX-MN 918 may be bound to a cellular membrane receptor (e.g., PSMA receptor 904). Once bound to the PSMA receptor 904, the targeting polypeptide CK-TGX-MN 918 may be cleaved by an extracellular protein (e.g., PSA 906a) at step 930. Once cleaved by PSA 906 at the glutamine (Q)—serine (S) bond, the dipeptide CK-TGX-MN conjugate 912 may then be internalized by peptide transporter 908 into the cell 901, as shown in step 940. Once internalized by the peptide transported 908, the dipeptide CK-TGX-MN conjugate 912 may be cleaved by dendritic cell-derived protein kinase (DPK) 914 to separate CK-TGX-MN payload 916 from the dipeptide so that CK-TGX-MN can inhibit the PKI3 pathway in cell 901 to kill the cell 901, thus effectively stopping tumor growth.

Additionally, or alternatively, polypeptide CK-TGX-MN 918 may enter the cell via endocytosis (e.g., potocytosis, receptor-mediated endocytosis, and so forth). For example, at step 950, the depicted CK-TGX-MN conjugate can bind to a cellular membrane bound receptor (e.g., PSMA receptor). Once bound, the CK-TGX-MN conjugate transferred into the cell via endocytosis. Once in the cytosol, an intracellular protein (e.g., PSA 906b) may cleave the CK-TGX-MN conjugate between the substrate (e.g., substrate 810 of FIG. 8) and dipeptide (e.g., dipeptide 804 of FIG. 8). The dipeptide CK-TGX-MN conjugate 912 may be cleaved by dendritic cell-derived protein kinase (DPK) 914 to separate CK-TGX-MN payload 916 from the dipeptide so that CK-TGX-MN can inhibit the PKI3 pathway in cell 901 to kill the cell 901, thus effectively stopping tumor growth.

As used throughout this description and in the claims originally presented and as may be presented at any time in this application and in any application claiming priority to this application, when a range of dimensions is presented as being “between” two stated dimension, the range is intended to include the stated dimensions. As such, a range presented as being ‘between x and y’ is intended to encompass and include x and y as part of the presented range); or a combination thereof.

Aspects of the present disclosure have been described with the intent to be illustrative rather than restrictive. Alternative aspects will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Not all steps listed in the various figures need be carried out in the specific order described.

As used herein and in connection with the claims listed hereinafter, the terminology “any of clauses” or similar variations of said terminology is intended to be interpreted such that features of claims/clauses may be combined in any combination. For example, an exemplary clause 4 may indicate the method/apparatus of any of clauses 1 through 3, which is intended to be interpreted such that features of clause 1 and clause 4 may be combined, elements of clause 2 and clause 4 may be combined, elements of clause 3 and 4 may be combined, elements of clauses 1, 2, and 4 may be combined, elements of clauses 2, 3, and 4 may be combined, elements of clauses 1, 2, 3, and 4 may be combined, and/or other variations. Further, the terminology “any of clauses” or similar variations of said terminology is intended to include “any one of clauses” or other variations of such terminology, as indicated by some of the examples provided above.

Clause 1. A therapeutic compound of formula (I) having oncological treatment properties by targeting phosphoinositide 3-kinase p110β isoform (PI3Kβ):

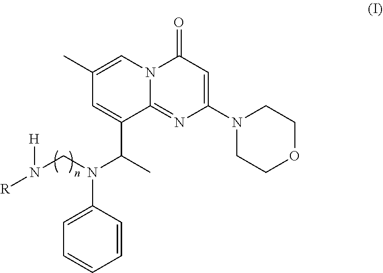

Clause 2. The compound of clause 1, wherein R is H or a substituent alkyl group.

Clause 3. The compound of clause 1, wherein R comprises an amino acid Clause 4. The compound of clause 1, wherein R comprises a dipeptide Clause 5. The compound of clause 1, wherein R comprises a peptide chain.

Clause 6. The compound of any of clauses 1, 3, 4 or 5, wherein R comprises a targeting compound.

Clause 7. The compound of clause 6, wherein the targeting compound comprises a linker.

Clause 8. The compound of clause 7, wherein the targeting compound further comprises a carrier.

Clause 9. The compound of any one of clauses 1-8, wherein “n” is an integer between 1 and 6.

Clause 10. A composition having oncological treatment properties by targeting phosphoinositide 3-kinase p110β isoform (PI3Kβ), the composition comprising: a compound of formula (I); and a linker.

Clause 11. The composition of clause 10, wherein the linker is R, and wherein the linker is a cleavable linker or a non-cleavable linker.

Clause 12. The composition of clauses 10 or 11, wherein the linker comprises a protease substrate.

Clause 13. The composition of any of clauses 10 through 12, further comprising a carrier.

Clause 14. The composition of clause 13, wherein the carrier is one of an aptamer, a peptide sequence, a small molecule, a protein sequence, or an antibody.

Clause 15. The composition of clause 13, wherein the linker connects the carrier to the compound.

Clause 16. The composition of any of clauses 13 through 15, wherein the carrier is an inhibitor of a cellular membrane protein expressed by a tumor cell.

Clause 17. The composition of any of clauses 11 through 16, wherein the linker further comprises a biologically compatible polymer, a substituent alkyl group, or a polyethylene glycol chain having the formula (OCH₂CH₂)ₓ wherein “x” is an integer between 4 and 182.

Clause 18. The composition of clause 17, wherein “x” is at least 5.

Clause 19. The composition of any one of clauses 10 through 18, wherein “n” is an integer between 1 and 6.

Clause 20. A method for treating an oncological condition of a subject by targeting phosphoinositide 3-kinase p110 isoform (PI3Kβ) in a tumor cell, the method comprising: administering a predefined dose of a medication formulation to the subject, wherein the medication formulation comprises a predetermined concentration of a compound of formula (I).

Clause 21. The method of clause 20, wherein the medication formulation is suitable for IV administration.

Clause 22. The method of clause 20, wherein the medication formulation is suitable for oral administration.

What is claimed is:

1. A therapeutic compound of formula (I) having oncological treatment properties by targeting phosphoinositide 3-kinase p110β isoform (PI3Kβ):

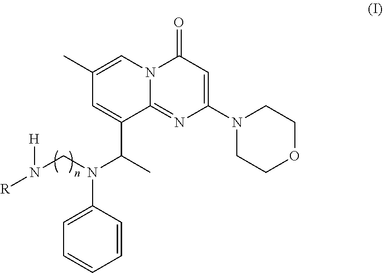

2. The compound of claim 1, wherein n is an integer between 1 and 6.

3. The compound of claim 1, wherein R is H or a substituent alkyl group.

4. The compound of claim 1, wherein R comprises an amino acid.

5. The compound of claim 1, wherein R comprises a dipeptide.

6. The compound of claim 1, wherein R comprises a peptide chain.

7. The compound of claim 1, wherein R comprises a targeting compound.

8. The compound of claim 6, wherein the targeting compound comprises a linker.

9. The compound of claim 7, wherein the targeting compound further comprises a carrier.

10. A composition having oncological treatment properties by targeting phosphoinositide 3-kinase p1103 isoform (PI3Kβ), the composition comprising: a compound of formula (I); and a linker.

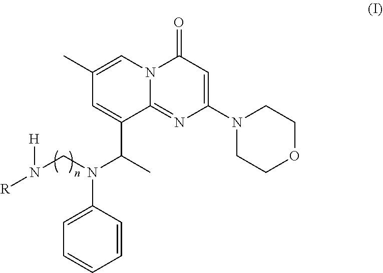

11. The composition of claim 10, wherein the linker is R, and wherein the linker is a cleavable linker or a non-cleavable linker.

12. The composition of claim 11, wherein the linker comprises a protease substrate.

13. The composition of claim 11, further comprising a carrier.

14. The composition of claim 13, wherein the carrier is one of an aptamer, a peptide sequence, a small molecule, a protein sequence, or an antibody.

15. The composition of claim 13, wherein the linker connects the carrier to the compound.

16. The composition of claim 15, wherein the carrier is an inhibitor of a cellular membrane protein expressed by a tumor cell.

17. The composition of claim 15, wherein the linker further comprises a biologically compatible polymer, a substituent alkyl group, an alkyl chain, or a polyethylene glycol chain having the formula (OCH2CH2)ₓ, wherein x is an integer between 4 and 182.

18. A method for treating an oncological condition of a subject by targeting phosphoinositide 3-kinase p110 isoform (PI3Kβ) in a tumor cell, the method comprising: administering a predefined dose of a medication formulation to the subject, wherein the medication formulation comprises a predetermined concentration of a compound of formula (I).

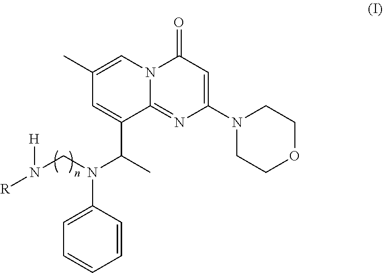

19. The method of claim 18, wherein the medication formulation is suitable for IV administration.

20. The method of claim 18, wherein the medication formulation is suitable for oral administration.
